(19) United States
(12) Patent Application Publication
(10) Pub. No.: US 2024/0261290 A1
(43) Pub. Date: Aug. 8, 2024

(54) PHARMACEUTICAL COMPOSITION COMPRISING 7-(4-CHLOROBENZYL)-1-(3-HYDROXYPROPYL)-3-METHYL-8-(3-(TRIFLUOROMETHOXY)-PHENOXY)-3,7-DIHYDRO-1H-PURINE-2,6-DIONE

(71) Applicants: Boehringer Ingelheim International GmbH, Ingelheim Am Rhein (DE); Hydra Biosciences, LLC, Belmont, MA (US)

(72) Inventors: Hans-Juergen Martin, Ingelheim Am Rhein (DE); Eva Epple, Ingelheim am Rhein (DE); Thomas Hennes, Ingelheim am Rhein (DE); Ragna Saila Maria Hoffmann, Ingelheim am Rhein (DE); Kerstin Julia Schaefer, Ingelheim am Rhein (DE); Claudia Weiss, Biberach an der Riss (DE)

(73) Assignees: Boehringer Ingelheim International GmbH, Ingelheim Am Rhein (DE); Hydra Biosciences, LLC, Belmont, MA (US)

(21) Appl. No.: 18/434,342

(22) Filed: Feb. 6, 2024

(30) Foreign Application Priority Data
Feb. 6, 2023 (EP) EP 23 155 058.3

(51) Int. Cl. A61K 31/522; A61K 9/00; A61K 9/20; A61K 9/28

(52) U.S. Cl. CPC A61K 31/522; A61K 9/0053; A61K 9/2013; A61K 9/2018; A61K 9/2054; A61K 9/2866

(57) ABSTRACT

The present invention relates to a novel solid oral pharmaceutical composition comprising 7-(4-chlorobenzyl)-1-(3-hydroxypropyl)-3-methyl-8-(3-(trifluoromethoxy)-phenoxy)-3,7-dihydro-1H-purine-2,6-dione (Compound 1) as pharmaceutically active compound, a method for preparing the same and its use as a medicament. The novel solid oral pharmaceutical composition comprises Compound 1, a sugar alcohol and at least one further pharmaceutically acceptable excipient.

### FIELD OF THE INVENTION

The present invention relates to a novel solid oral pharmaceutical composition comprising 7-(4-chlorobenzyl)-1-(3-hydroxypropyl)-3-methyl-8-(3-(trifluoromethoxy)-phenoxy)-3,7-dihydro-1H-purine-2,6-dione (Compound 1) as pharmaceutically active compound, a method for preparing the same and its use as a medicament.

### BACKGROUND OF THE INVENTION

Compound 1 is a TRPC4/5 cation channel inhibitor. TRPC5 inhibitors are for example known from WO2014/143799. TRPC5 inhibitors modulate the function of TRPC5 by inhibiting a TRPC5-mediated ion flux or by inhibiting the inward current, the outward current, or both currents mediated by TRPC5. Based on their TRPC5-inhibiting activity they can be used for treating conditions such as a neuropsychiatric disorder, a neurodegenerative disorder, nephropathy, and seizure disorder.

Compound 1 exhibits a high lipophilicity (logP: 5.2) and a low aqueous solubility under acidic to neutral conditions. The oral bioavailability of Compound 1 thus is prone to considerable inter- and intra-individual fluctuations depending on patients' fasting state when incorporated in conventional pharmaceutical dosage forms for oral administration.

The aim of the present invention is to provide a suitable pharmaceutical composition comprising Compound 1 as pharmaceutically active compound allowing for the oral administration of Compound 1 with a high and reproducible bioavailability.

### SUMMARY OF THE INVENTION

It was found that when incorporating Compound 1 in a sugar alcohol-based solid composition, a high oral bioavailability with a fed/fasted ratio of about 2 or below can be achieved.

The following items are provided in accordance with the present invention:
1. Solid oral pharmaceutical composition comprising Compound 1, a sugar alcohol and at least one further pharmaceutically acceptable excipient.
2. The solid oral pharmaceutical composition of item 1, wherein the sugar alcohol is selected from mannitol, sorbitol, and xylitol.
3. The solid oral pharmaceutical composition of item 1, wherein the sugar alcohol is mannitol.
4. The solid oral pharmaceutical composition of item 1, wherein amount of the sugar alcohol is 40-80% [w/w].
5. The solid oral pharmaceutical composition of item 1, wherein the at least one further pharmaceutically acceptable excipient comprises one or more selected from binders, fillers, disintegrants, glidants, lubricants, wetting agents, surfactants and preservatives.
6. The solid oral pharmaceutical composition of item 1, wherein the at least one further pharmaceutically acceptable excipient comprises a binder.
7. The solid oral pharmaceutical composition of item 6, wherein the binder is a cellulose derivative.
8. The solid oral pharmaceutical composition of item 7, wherein the cellulose derivative is selected from methylcellulose, hydroxyethylcellulose and hydroxypropylcellulose.
9. The solid oral pharmaceutical composition of item 8, wherein the cellulose derivative is hydroxypropylcellulose.
10. The solid oral pharmaceutical composition of item 6, wherein amount of the binder is 1-5% [w/w].
11. The solid oral pharmaceutical composition of item 6, wherein the sugar alcohol is mannitol and the binder is hydroxypropylcellulose.
12. The solid oral pharmaceutical composition of item 1, wherein the composition is a granulate, a hard capsule or a tablet.
13. The solid oral pharmaceutical composition of item 12, wherein the composition is a tablet, which is optionally coated.
14. The solid oral pharmaceutical composition of item 1, wherein Compound 1 is present in an amount of 5-50% [w/w].
15. The solid oral pharmaceutical composition of item 1, wherein Compound 1 is present as sole active ingredient.
16. The solid oral pharmaceutical composition of item 1, wherein Compound 1 is incorporated in form of particles having a particle size of D90<100 μm.
17. The solid oral pharmaceutical composition of item 1, wherein Compound 1 is incorporated in form of particles having a particle size of D50<500 nm.
18. The solid oral pharmaceutical composition of item 1, wherein the composition comprises:
   5-50% [w/w] Compound 1,
   40-80% [w/w] sugar alcohol, and
   one or more further pharmaceutically acceptable excipient(s), wherein Compound 1, the sugar alcohol, and the one or more further pharmaceutically acceptable excipient(s) add up to a total of 100% [w/w].
19. The solid oral pharmaceutical composition of item 18, wherein the sugar alcohol is mannitol.
20. The solid oral pharmaceutical composition of item 1, wherein the composition is a tablet, and the tablet core comprises:
5-50% [w/w] Compound 1 (incorporated in form of micronized or nanosized particles),
40-80% [w/w] sugar alcohol,
0-10% [w/w] binder,
0-30% [w/w] filler,
0-10% [w/w] disintegrant,
0-5% [w/w] glidant,
0-5% [w/w] lubricant,
   and 0-1% [w/w] each of a wetting agent, a surfactant and a preservative, wherein the amount of Compound 1 and the respective excipients adds up to a total of 100% [w/w] and,
   wherein the tablet optionally is coated.
21. The solid oral pharmaceutical composition of item 1, wherein the composition is a tablet, and the tablet core comprises:
5-50% [w/w] Compound 1 (incorporated in form of micronized or nanosized particles),
40-80% [w/w] manhitol
0-10% [w/w] hydroxpropylceiiuiose,
0-30% [w/w] microcrystalline cellulose,
0-10% [w/w] croscarmellose sodium,
0-5% [w/w] colloidal silicon dioxide,
0-5% [w/w] magnesium stearate,
0-1% [w/w] sodium laurilsulfate,
0-1% [w/w] Polysorbate 80, and
0-1% [w/w] Parahydroxybenzoate,
   wherein the amount of Compound 1 and the respective excipients adds up to a total of 100% [w/w] and, wherein the tablet optionally is coated.
22. The solid oral pharmaceutical composition of any of items 13, 20 and 21, wherein the tablet is coated, and the coating is free of titanium dioxide.
23. The solid oral pharmaceutical composition of any of items 1-22 prepared by a wet granulation method, wherein Compound 1 preferably is suspended in the granulation liquid.
24. A method for preparing the solid oral pharmaceutical composition of any of items 1-22, wherein the method is a wet granulation method.
25. The method of item 24, wherein the method is a fluid bed granulation method and Compound 1 is suspended in the granulation liquid.
26. The method of item 24, wherein the method comprises the following steps:
   preparing a granulation liquid containing Compound 1 in micronized or nanosized form, a first portion of the sugar alcohol, and the at least one further pharmaceutically acceptable excipient, preferably a binder;
   granulating the second portion of the sugar alcohol with the granulation liquid in a suitable fluid-bed granulator;
   drying the wet granules in the fluid-bed granulator to obtain dry granules;
   optionally, screening the dried granules with a suitable screen;
   combining the granules with one or more selected from fillers, disintegrants, glidants, and lubricants to obtain a premix, optionally screening the premix, and blending the optionally screened premix to obtain a blend;
   compressing the blend into tablet cores using a suitable tablet press; and
   further optionally, coating the tablet cores with a previously prepared film-coating suspension by spraying to produce Compound 1-containing film-coated tablets.
27. The method of item 26, wherein the at least one further pharmaceutically acceptable excipient contained in the granulation liquid includes a binder and one or more of a wetting agent, a surfactant and a preservative, preferably a wetting agent and/or a surfactant.
28. Granulation liquid containing Compound 1 in micronized or nanosized form, a sugar alcohol, at least one further pharmaceutically acceptable excipient, preferably a binder, and, optionally, one or more of a wetting agent, a surfactant and a preservative, and water.
29. The solid oral pharmaceutical composition according to any of items 1-23 for use as a medicament.
30. The solid oral pharmaceutical composition according to any of items 1-23 for use in the treatment of conditions such as a neuropsychiatric disorder, e.g., major depressive disorder (MDD) and post-traumatic stress disorder (PTSD), a neurodegenerative disorder, nephropathy, and seizure disorder.

### BRIEF DESCRIPTION OF THE DRAWINGS

FIG. 1 schematically shows the preparation method of the solid oral pharmaceutical compositions of the present invention.

FIG. 2 shows a flow chart displaying the preparation method for example formulation A.

FIG. 3 shows a flow chart displaying the preparation method for example formulations B and C.

FIG. 4 shows a flow chart displaying the preparation method for example formulation D.

FIG. 5 shows the results of comparative dissolution testing of the 50 mg tablet core and the 50 mg final film-coated tablet of example formulation B.

FIG. 6 shows the X-ray powder diffraction (XRPD) diagram of Compound 1.

### DETAILED DESCRIPTION OF THE INVENTION

The present invention is based on the finding that when formulating Compound 1 in a sugar alcohol-based solid composition, a high oral bioavailability of Compound 1 can be achieved while at the same time significantly reducing the influence of food effects. It is well known that the administration of a drug together with food may change its bioavailability. Basically, food effects on the bioavailability of a drug are most pronounced when the drug is taken shortly after a meal. Food effects can be expressed by determining the fed/fasted ratio, i.e., by determining the bioavailability under fed and fasted conditions and comparing the results by forming the respective quotient.

The fed/fasted ratio for the compositions of the present invention is below 3, preferably below 2.5 and more preferably below 2.1.

To achieve the above-described beneficial effects, Compound 1 is formulated according to the present invention in a sugar alcohol-based solid composition for oral use.

In a first aspect, the present invention thus relates to a solid oral pharmaceutical composition comprising Compound 1, a sugar alcohol and at least one further pharmaceutically acceptable excipient.

The term “Compound 1” as employed herein comprises the anhydrate as well as hydrates and solvates of Compound 1 in amorphous or crystalline form, respectively.

In one embodiment, Compound 1 used for preparing the solid oral pharmaceutical composition of the present invention has a particle size of D90<100 μm, preferably of D90≤ 15 μm. Corresponding particles of Compound 1 are herein also referred to as “micronized”.

In another embodiment, Compound 1 used for preparing the solid oral pharmaceutical composition of the present invention has a particle size of D50<500 nm, preferably of D50<200 nm. Corresponding particles of Compound 1 are herein also referred to as “nanosized”.

In a preferred embodiment, Compound 1 used for preparing the solid oral pharmaceutical composition of the present invention is micronized.

In another preferred embodiment, Compound 1 is present as sole active ingredient in the solid oral pharmaceutical composition of the present invention.

In a preferred embodiment, Compound 1 is present in the solid oral pharmaceutical composition of the present invention in an amount of 5-50% [w/w], preferably 5-30% [w/w] and more preferably 5-25% [w/w].

Where the amount is indicated as “% [w/w]”, this relates here and in the following to the total amount of the solid oral pharmaceutical composition of the present invention as such, i.e., without any potential coating.

In a preferred embodiment, the sugar alcohol for use in the present invention is selected from mannitol, sorbitol, and xylitol. Preferably, the sugar alcohol is mannitol.

In a preferred embodiment, the sugar alcohol is present in the solid oral pharmaceutical composition of the present invention in an amount of 40-80% [w/w] and preferably 50-70% [w/w].

In one embodiment, the at least one further pharmaceutically acceptable excipient of the solid oral pharmaceutical composition of the present invention comprises one or more selected from binders, fillers, disintegrants, glidants, lubricants, wetting agents, surfactants and preservatives.

In a preferred embodiment, the at least one further pharmaceutically acceptable excipient of the solid oral pharmaceutical composition of the present invention comprises a binder.

In another preferred embodiment, the at least one further pharmaceutically acceptable excipient of the pharmaceutical composition of the present invention comprises a binder, a wetting agent and/or a surfactant.

Binders suitable for use in the solid oral pharmaceutical composition of the present invention may be selected from polyvinylpyrrolidone (PVP), copovidone, starch, cellulose derivatives or polyethylene glycol. The binder preferably is a cellulose derivative, and more preferably methylcellulose, hydroxyethylcellulose or hydroxypropylcellulose. Most preferably, the binder is hydroxypropylcellulose.

The binder(s) used as at least one further pharmaceutically acceptable excipient is/are preferably present in the solid oral pharmaceutical composition of the present invention in an amount of 0-10% [w/w], more preferably 1-5% [w/w] and most preferably 2-3% [w/w].

Fillers suitable for use in the solid oral pharmaceutical composition of the present invention may be selected from inorganic phosphates, e.g., dibasic calcium phosphate, lactose, such as lactose monohydrate or water-free lactose, dextrose, saccharose, maltodextrin, isomalt, and microcrystalline cellulose. Preferably, the filler is microcrystalline cellulose.

The filler(s) used as at least one further pharmaceutically acceptable excipient is/are preferably present in the solid oral pharmaceutical composition of the present invention in an amount of 0-30% [w/w], more preferably 5-25% [w/w] and most preferably 10-20% [w/w].

Disintegrants suitable for use in the solid oral pharmaceutical composition of the present invention may be selected from crospovidone, sodium starch glycolate, alginates, pregelatinized starch, and croscarmellose sodium. Preferably, the disintegrant is croscarmellose sodium.

The disintegrant(s) used as at least one further pharmaceutically acceptable excipient is/are preferably present in the solid oral pharmaceutical composition of the present invention in an amount of 0-10% [w/w], more preferably 1-5% [w/w] and most preferably 2-3% [w/w].

Glidants suitable for use in the solid oral pharmaceutical composition of the present invention may be selected from cornstarch and colloidal silicon dioxide. Preferably, the glidant is colloidal silicon dioxide.

The glidant(s) used as at least one further pharmaceutically acceptable excipient is/are preferably present in the solid oral pharmaceutical composition of the present invention in an amount of 0-5% [w/w] or 1-2% [w/w].

In a preferred embodiment, the solid oral pharmaceutical composition of the present invention does not contain a glidant.

Lubricants suitable for use in the solid oral pharmaceutical composition of the present invention may be selected from talc, alkali or earth alkali salts of stearic acid, e.g., magnesium stearate, and sodium stearyl fumarate. Preferably, the lubricant is magnesium stearate.

The lubricant(s) used as at least one further pharmaceutically acceptable excipient is/are preferably present in the solid oral pharmaceutical composition of the present invention in an amount of 0-5% [w/w] and more preferably 1-2% [w/w].

The at least one further pharmaceutically acceptable excipient of the solid oral pharmaceutical composition of the present invention may additionally comprise a wetting agent, a surfactant or a combination thereof. Preferably, the wetting agent is sodium laurilsulfate. The surfactant can be selected from any of Polysorbate 80 and Poloxamer 188. Preferably, the surfactant is Polysorbate 80.

In a preferred embodiment, the solid oral pharmaceutical composition of the present invention comprises a wetting agent, preferably sodium laurilsulfate.

In another embodiment, sodium laurilsulfate and Polysorbate 80 are used in combination in the solid oral pharmaceutical composition of the present invention.

Further, the at least one further pharmaceutically acceptable excipient of the solid oral pharmaceutical composition of the present invention can optionally comprise a preservative, preferably parahydroxybenzoate.

When used in the solid oral pharmaceutical composition of the present invention, the wetting agent, the surfactant and the preservative are preferably present in an amount of no more than 1% [w/w] each, preferably of no more than 0.1% [w/w] each and further preferred of 0.01-1% [w/w] or 0.01-0.1% [w/w] each.

In a preferred embodiment, the solid oral pharmaceutical composition of the present invention comprises
5-50% [w/w] Compound 1 (incorporated in form of micronized or nanosized particles),
40-80% [w/w] sugar alcohol, and

optionally one or more further pharmaceutically acceptable excipient(s), wherein Compound 1, the sugar alcohol, and the one or more further pharmaceutically acceptable excipient(s) add up to a total of 100% [w/w].

In another preferred embodiment, the solid oral pharmaceutical composition of the present invention comprises
5-50% [w/w] Compound 1 (incorporated in form of micronized or nanosized particles),
40-80% [w/w] sugar alcohol,
0-10% [w/w] binder,
0-30% [w/w] filler,
0-10% [w/w] disintegrant,
0-5% [w/w] glidant,
0-5% [w/w] lubricant,

and 0-1% [w/w] each of a wetting agent, a surfactant and a preservative, wherein the amount of Compound 1 and the respective excipients adds up to a total of 100% [w/w].

In further preferred embodiments, the solid oral pharmaceutical composition of the present invention comprises
5-30% [w/w] Compound 1 (incorporated in form of micronized particles),
50-70% [w/w] sugar alcohol,
1-5% [w/w] binder,
5-25% [w/w] filler,
1-5% [w/w] disintegrant,
0-5% [w/w] glidant,
1-2% [w/w] lubricant,
   and 0-1% [w/w] each of a wetting agent, a surfactant and a preservative, wherein the amount of Compound 1 and the respective excipients adds up to a total of 100% [w/w]; or
5-30% [w/w] Compound 1 (incorporated in form of micronized particles),
50-70% [w/w] sugar alcohol,
1-5% [w/w] binder,
5-25% [w/w] filler,
1-5% [w/w] disintegrant,
0-5% [w/w] glidant,
1-2% [w/w] lubricant,
   and 0-1% [w/w] each of a wetting agent, a surfactant and a preservative, wherein the amount of Compound 1 and the respective excipients adds up to a total of 100% [w/w]; or
5-30% [w/w] Compound 1 (incorporated in form of nanosized particles),
50-70% [w/w] sugar alcohol,
1-5% [w/w] binder,
5-25% [w/w] filler,
1-5% [w/w] disintegrant,
0-5% [w/w] glidant,
1-2% [w/w] lubricant,
   and 0.01-1% [w/w] each of a wetting agent, a surfactant and a preservative, wherein the amount of Compound 1 and the respective excipients adds up to a total of 100% [w/w]; or
5-30% [w/w] Compound 1 (incorporated in form of nanosized particles),
50-70% [w/w] sugar alcohol,
1-5% [w/w] binder,
5-25% [w/w] filler,
1-5% [w/w] disintegrant,
0-5% [w/w] glidant,
1-2% [w/w] lubricant,
   and 0.01-1% [w/w] each of a wetting agent, a surfactant and a preservative, wherein the amount of Compound 1 and the respective excipients adds up to a total of 100% [w/w].

Preferably, the sugar alcohol in any of the above compositions is mannitol.

In still another preferred embodiment, the solid oral pharmaceutical composition of the present invention comprises
5-50% [w/w] Compound 1 (incorporated in form of micronized or nanosized particles),
40-80% [w/w] manhitol
0-10% [w/w] hydroxpropylcellulose,
0-30% [w/w] microcrystalline cellulose,
0-10% [w/w] croscarmellose sodium,
0-5% [w/w] colloidal silicon dioxide,
0-5% [w/w] magnesium stearate,
0-1% [w/w] sodium laurilsulfate,
0-1% [w/w] Polysorbate 80, and
0-1% [w/w] Parahydroxybenzoate,
   wherein the amount of Compound 1 and the respective excipients adds up to a total of 100% [w/w].

In further preferred embodiments, the solid oral pharmaceutical composition of the present invention comprises
5-30% [w/w] Compound 1 (incorporated in form of micronized particles),
50-70% [w/w] manhitol
1-5% [w/w] hydroxpropylcellulose,
5-25% [w/w] microcrystalline cellulose,
1-5% [w/w] croscarmellose sodium,
0-5% [w/w] colloidal silicon dioxide,
1-2% [w/w] magnesium stearate,
0-0.1% [w/w] sodium laurilsulfate,
0-0.1% [w/w] Polysorbate 80, and
0-0.1% [w/w] Parahydroxybenzoate,
   wherein the amount of Compound 1 and the respective excipients adds up to a total of 100% [w/w]; or
5-30% [w/w] Compound 1 (incorporated in form of micronized particles),
40-80% [w/w] manhitol
0-10% [w/w] hydroxpropylcellulose,
0-30% [w/w] microcrystalline cellulose,
0-10% [w/w] croscarmellose sodium,
0-5% [w/w] colloidal silicon dioxide,
0-5% [w/w] magnesium stearate,
0-1% [w/w] sodium laurilsulfate,
0-1% [w/w] Polysorbate 80, and
0-1% [w/w] Parahydroxybenzoate,
   wherein the amount of Compound 1 and the respective excipients adds up to a total of 100% [w/w]; or
5-30% [w/w] Compound 1 (incorporated in form of nanosized particles),
50-70% [w/w] manhitol
1-5% [w/w] hydroxpropylcellulose,
5-25% [w/w] microcrystalline cellulose,
1-5% [w/w] croscarmellose sodium,
0-5% [w/w] colloidal silicon dioxide,
1-2% [w/w] magnesium stearate,
0.01-0.1% [w/w] sodium laurilsulfate,
0.01-0.1% [w/w] Polysorbate 80, and
0.01-0.1% [w/w] Parahydroxybenzoate,
   wherein the amount of Compound 1 and the respective excipients adds up to a total of 100% [w/w]; or
5-30% [w/w] Compound 1 (incorporated in form of nanosized particles),
50-70% [w/w] manhitol
1-5% [w/w] hydroxpropylcellulose,
5-25% [w/w] microcrystalline cellulose,
1-5% [w/w] croscarmellose sodium,
1-2% [w/w] colloidal silicon dioxide,
1-2% [w/w] magnesium stearate,
0.01-0.1% [w/w] sodium laurilsulfate,
0.01-0.1% [w/w] Polysorbate 80, and
0.01-0.1% [w/w] Parahydroxybenzoate,
   wherein the amount of Compound 1 and the respective excipients adds up to a total of 100% [w/w].

The solid oral pharmaceutical composition of the present invention preferably is a granulate, a hard capsule or a tablet, and most preferably a tablet.

In one embodiment, the solid oral pharmaceutical composition of the present invention is a tablet, which is optionally coated.

A corresponding coating composition preferably comprises one or more film-forming polymer(s) and one or more pharmaceutically acceptable excipient(s).

Suitable film-forming polymers for use in the coating composition include, for example, hydroxypropylmethylcellulose (Hypromellose), ethylcellulose, methylcellulose, hydroxyethylcellulose, hydroxypropylcellulose, sodiumcarboxymethylcellulose, celluloseacetate, hydroxypropylmethylcellulose phthalate, celluloseacetate trimellitate, methacrylic acid copolymers, e.g., Eudragit®, polyvinylpyrrolidone, polyvinylalcohol, macrogol-poly(vinyl alcohol) graft copolymer, polyethylene glycol, or mixtures thereof. Other film-forming polymers which are known in the art may also be used.

In a preferred embodiment, the film-forming polymer is hydroxypropylmethylcellulose. In another preferred embodiment, hydroxypropylmethylcellulose is used in combination with hydroxypropylcellulose as film-forming polymer.

Suitable pharmaceutically acceptable excipients for use in the coating composition include, for example, a softener, for example a polyethyleneglycol, GMCC (a mixture of monoglycerides and diglycerides of caprylic and capric acids), medium chain triglycerides (MCTs) and isomalt, an anti-adhesive agent, for example talc, a pigment, for example titanium dioxide or calcium carbonate, and a dye, for example, iron oxide pigments.

An example of a film-coating composition for use in the present invention comprises hydroxypropylmethylcellulose, propylene glycol, talc, titanium dioxide and, optionally, iron oxide, e.g., iron oxide yellow and/or red.

In a preferred embodiment, the film-coating composition for use in the present invention is free of titanium dioxide. A titanium dioxide-free coating suitable for use in the present invention can for example comprise
hydroxypropylmethylcellulose, hydroxypropylcellulose, medium chain triglycerides, isomalt, calcium carbonate, and, optionally, iron oxide, e.g., iron oxide yellow and/or red;
hydroxypropylmethylcellulose, hydroxypropylcellulose, propylene glycol, talc, calcium carbonate, and, optionally, iron oxide, e.g., iron oxide yellow and/or red; or
hydroxypropylmethylcellulose, propylene glycol, talc, calcium carbonate, and, optionally, iron oxide, e.g., iron oxide yellow and/or red.

In one embodiment, commercially available film-coating compositions such as Opadry® or Aquapolish® P can be used for coating the solid oral pharmaceutical composition of the present invention.

The coating can be applied by known film-coating techniques, i.e., spray coating, fluid bed coating or dip coating.

Examples of solvents that can be used for preparing the initial coating solution of the coating composition employed for the film-coating step are selected from methyl alcohol, ethyl alcohol, isopropyl alcohol, n-butyl alcohol, acetone, acetonitrile, chloroform, methylene chloride, water, or mixtures thereof.

The coating composition described herein generally does not contain Compound 1.

In an alternative embodiment, the coating composition can contain at least some of the Compound 1, while the remaining amount of Compound 1 is part of the tablet core.

In a preferred embodiment, the solid oral pharmaceutical composition of the present invention is an immediate release film-coated tablet.

The following are preferred embodiments of the solid oral pharmaceutical composition of the present invention in form of tablets being optionally coated:
a)
5-30% [w/w] Compound 1 (incorporated in form of nanosized particles),
50-70% [w/w] manhitol
1-5% [w/w] hydroxpropylcellulose,
5-25% [w/w] microcrystalline cellulose,
1-5% [w/w] croscarmellose sodium,
1-2% [w/w] colloidal silicon dioxide,
1-2% [w/w] magnesium stearate,
0.01-0.1% [w/w] sodium laurilsulfate,
0.01-0.1% [w/w] Polysorbate 80, and
0.01-0.1% [w/w] Parahydroxybenzoate,
b)
5-30% [w/w] Compound 1 (incorporated in form of nanosized particles),
50-70% [w/w] manhitol
1-5% [w/w] hydroxpropylcellulose,
5-25% [w/w] microcrystalline cellulose,
1-5% [w/w] croscarmellose sodium,
1-2% [w/w] colloidal silicon dioxide,
1-2% [w/w] magnesium stearate,
0.01-0.1% [w/w] sodium laurilsulfate,
0.01-0.1% [w/w] Polysorbate 80, and
0.01-0.1% [w/w] Parahydroxybenzoate,
c)
7.46% [w/w] Compound 1 (incorporated in form of micronized particles)
3.00% [w/w] Hydroxpropyl cellulose
0.10% [w/w] Sodium laurilsulfate
69.44% [w/w] Manhitol
16.50% [w/w] Cellulose microcrystalline
2.50% [w/w] Croscarmellose sodium,
1.00% [w/w] Magnesium stearate,
d)
25.00% [w/w] Compound 1 (incorporated in form of microsized particles),
51.90% [w/w] Manhitol
3.00% [w/w] Hydroxpropyl cellulose,
0.10% [w/w] Sodium laurilsulfate,
16.50% [w/w] Cellulose, microcrystalline
2.50% [w/w] Croscarmellose sodium,
1.00% [w/w] Magnesium stearate,
e)
7.46[w/w] Compound 1 (incorporated in form of nanosized particles),
3.00% [w/w] Hydroxpropyl cellulose
0.02% [w/w] Sodium laurilsulfate
0.02% [w/w] Polysorbat
0.07% [w/w] Methyl parahydroxybenzoate
69.44% [w/w] Manhitol
16.50% [w/w] Cellulose microcrystalline
2.50% [w/w] Croscarmellose sodium,
1.00% [w/w] Magnesium stearate,

In a second aspect, the present invention relates to a method for preparing the solid oral pharmaceutical composition of the present invention comprising Compound 1, a sugar alcohol and at least one further pharmaceutically acceptable excipient.

The preparation method of the present invention is a wet granulation method, preferably a fluid-bed granulation method.

In a particularly preferred embodiment, the preparation method of the present invention is a fluid-bed granulation method, wherein Compound 1 is suspended in the granulation liquid.

Accordingly, the solid oral pharmaceutical composition of the present invention is prepared by a wet granulation method, preferably a fluid-bed granulation method.

In a preferred embodiment, the solid oral pharmaceutical composition of the present invention is prepared by a fluid bed granulation method, wherein Compound 1 is suspended in the granulation liquid.

In one embodiment, the preparation method of the present invention comprises the following steps:
preparing a granulation liquid containing Compound 1 in micronized or nanosized form, a first portion of the sugar alcohol, and the at least one further pharmaceutically acceptable excipient, preferably a binder;
granulating the second portion of the sugar alcohol with the granulation liquid in a suitable fluid-bed granulator;
drying the wet granules in the fluid-bed granulator to obtain dry granules;
optionally, screening the dried granules with a suitable screen;
combining the granules with one or more selected from fillers, disintegrants, glidants, and lubricants to obtain a premix, optionally screening the premix, and blending the optionally screened premix to obtain a blend;
compressing the blend into tablet cores using a suitable tablet press; and
further optionally, coating the tablet cores with a previously prepared film-coating suspension by spraying to produce Compound 1-containing film-coated tablets.

In another embodiment, the preparation method of the present invention comprises the following steps:
a first portion of binder, one or more of a wetting agent, a surfactant and a preservative, preferably a wetting agent and/or a surfactant, are added to a solvent and mixed to produce a vehicle;
micronized Compound 1 is added to the vehicle and stirred to obtain a suspension;
the suspension is milled in a wet milling process to obtain a suspension of nanosized Compound 1;
a second portion of binder and the solvent are mixed to obtain a binder liquid;
while stirring the suspension of nanosized Compound 1, the binder liquid is added;
afterwards a first portion of the sugar alcohol is added to obtain a granulation liquid;
a second portion of the sugar alcohol is pre-heated and then granulated with the granulation liquid in a suitable fluid-bed granulator;
the wet granules are dried in the fluid-bed granulator to obtain dry granules; and
the dried granules are optionally screened with a suitable screen;
   or, alternatively:
binder, one or more of a wetting agent, a surfactant and a preservative, preferably a wetting agent and/or a surfactant, and a first portion of sugar alcohol (optionally pre-screened) are dissolved in a solvent;
afterwards, micronized Compound 1 is suspended therein to obtain a granulation liquid;
a second portion of the sugar alcohol is pre-heated and then granulated with the granulation liquid in a suitable fluid-bed granulator;
the wet granules are dried in the fluid-bed granulator to obtain dry granules; and
the dried granules are optionally screened with a suitable screen.

The obtained granules can further be processed to tablets by a direct compression process or as follows:
filler, disintegrant and the optionally pre-screened granules are blended to obtain a main blend;
the main blend and lubricant are blended to produce a final blend, wherein, preferably, a first portion of the main blend is blended with the lubricant and then the remainder of the main blend is added and blended, or the main blend is blended in one portion with the lubricant; and
the final blend is compressed into tablet cores using a suitable tablet press;
   or, alternatively:
filler, disintegrant and the optionally pre-screened granules are blended to obtain a blend;
lubricant is added to produce a main blend, wherein either a first portion of the blend is combined with the lubricant and then the remainder of the blend is added, or, preferably, the blend is combined in one portion with the lubricant;
the main blend is optionally screened, and
the optionally screened main blend is blended to obtain the final blend; and
the final blend is compressed into tablet cores using a suitable tablet press.

Without being bound by theory, it is assumed that the resulting tablets include Compound 1 layered on the sugar alcohol particles thereby forming a matrix with Compound 1 being embedded therein.

The tablets can further be film coated as follows:
a film-coating composition is dispersed in a solvent by stirring in a suitable mixing vessel to prepare a film-coating suspension; and
tablet cores are coated with the film-coating suspension by spraying to produce Compound 1-containing film-coated tablets.

Alternatively, the granules obtained by any of the above preparation methods can be processed to capsules by optionally blending the granules, for example with a filler, and then filling them into capsules.

Where reference is made in the present description to blending the respective materials, this may either including mixing them as such or screening them together.

The solvent used for preparing the granulation liquid is selected from methyl alcohol, ethyl alcohol, isopropyl alcohol, n-butyl alcohol, acetone, acetonitrile, chloroform, methylene chloride, water, or mixtures thereof. Preferably, the solvent is water.

The granulation liquid, in a preferred embodiment, contains Compound 1 in micronized or nanosized form, a sugar alcohol, at least one further pharmaceutically acceptable excipient, preferably a binder, and, optionally, one or more of a wetting agent, a surfactant and a preservative, and water.

Micronized Compound 1 can be prepared by jet milling. Nanosized Compound 1 can be prepared by wet milling of a previously prepared suspension of micronized Compound 1.

In a third aspect, the present invention relates to the use of the solid oral pharmaceutical composition of the present invention comprising Compound 1, a sugar alcohol and at least one further pharmaceutically acceptable excipient as a medicament.

In a preferred embodiment, the solid oral pharmaceutical composition of the present invention is for use in the treatment of conditions such as a neuropsychiatric disorder, e.g., major depressive disorder (MDD) and post-traumatic stress disorder (PTSD), a neurodegenerative disorder, nephropathy, and seizure disorder.

### EXAMPLES

The following examples serve to further illustrate the present invention; but the same should not be construed as limiting the scope of the invention disclosed herein.

### Example 1

Compound 1-containing immediate release film-coated tablet formulations were prepared in dose strengths of 5 mg, 25 mg, 50 mg, 75 mg, 100 mg, and 125 mg.

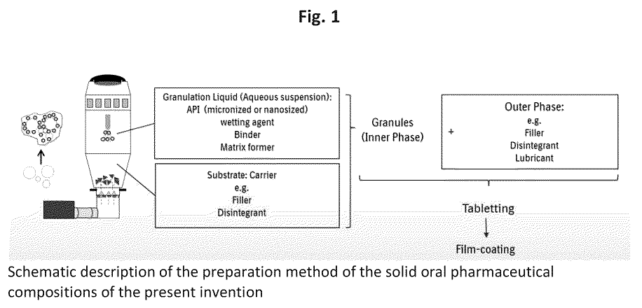

Tablets were prepared by wet-granulation using mannitol and microcrystalline cellulose as filler, magnesium stearate as lubricant, hydroxypropylcellulose as binder, and croscarmellose sodium as disintegrant, while Compound 1 was suspended in the granulation liquid. The preparation method is schematically shown in FIG. 1. Due to the excellent flowability of the obtained granules, adding a glidant, such as colloidal anhydrous silica, was not needed.

Four tablet formulations were prepared, formulation A, formulation B, formulation C, and formulation D having the following compositions:

TABLE 1
Composition formulation A

| | Dose strength | | | |
|---|---|---|---|---|
| | 5 mg | 25 mg | 50 mg | all |
| | Drug load (%, wt/wt) | | | |
| | | | | 7.46 |
| | 7.46 | 7.46 | 7.46 | [%, |
| Ingredient | [mg/tab] | [mg/tab] | [mg/tab] | wt/wt] |
| Compound 1 | 5.000 | 25.000 | 50.000 | 7.46 |
| (D50 < 200 nm) | | | | |
| Hydroxypropyl cellulose | 2.010 | 10.050 | 20.100 | 3.00 |
| Sodium laurilsulfate | 0.014 | 0.070 | 0.140 | 0.02 |
| Polysorbat 80 | 0.014 | 0.070 | 0.140 | 0.02 |
| Methyl | 0.047 | 0.235 | 0.470 | 0.07 |
| parahydroxybenzoate | | | | |
| Mannitol | 46.515 | 232.575 | 465.150 | 69.43 |
| Cellulose, microcrystalline | 11.055 | 55.275 | 110.550 | 16.50 |
| Croscarmellose sodium | 1.675 | 8.375 | 16.750 | 2.50 |
| Magnesium stearate | 0.670 | 3.350 | 6.700 | 1.00 |
| Sub-total Core | 67.000 | 335.000 | 670.000 | 100.00 |
| Film-coat Opadry ® red* | 3.000 | 9.000 | 14.000 | — |
| Total Film coated tablet | 70.000 | 344.000 | 684.000 | — |

*Composition film coat: Hypromellose 2910, Macrogol 6000, titanium dioxide, talc, iron oxide red

TABLE 2
Composition formulation B

| | Dose strength | | | |
|---|---|---|---|---|
| | 5 mg | 25 mg | 50 mg | all |
| | Drug load (%, wt/wt) | | | |
| | | | | 7.46 |
| | 7.46 | 7.46 | 7.46 | [%, |
| Ingredient | [mg/tab] | [mg/tab] | [mg/tab] | wt/wt] |
| Compound 1 | 5.000 | 25.000 | 50.000 | 7.46 |
| (D90 ≤ 15 μm) | | | | |
| Hydroxypropyl cellulose | 2.010 | 10.050 | 20.100 | 3.00 |
| Sodium laurilsulfate | 0.067 | 0.335 | 0.670 | 0.10 |
| Mannitol | 46.523 | 232.615 | 465.230 | 69.44 |
| Cellulose, microcrystalline | 11.055 | 55.275 | 110.550 | 16.50 |
| Croscarmellose sodium | 1.675 | 8.375 | 16.750 | 2.50 |
| Magnesium stearate | 0.670 | 3.350 | 6.700 | 1.00 |
| Sub-total Core | 67.000 | 335.000 | 670.000 | 100.00 |
| Film-coat Opadry ® red* | 3.000 | 9.000 | 14.000 | — |
| Total Film coated tablet | 70.000 | 344.000 | 684.000 | — |

*Composition film coat: Hypromellose 2910, Macrogol 6000, titanium dioxide, talc, iron oxide red

TABLE 3
Composition formulation C

Dose strength
100 mg
Drug load (%, wt/wt)

| | 25.00 | 25.00 |
|---|---|---|
| Ingredient | [mg/tab] | [%, wt/wt] |
| Compound 1 (D90 ≤ 15 μm) | 100.00 | 25.00 |
| Hydroxypropyl cellulose | 12.00 | 3.00 |
| Sodium laurilsulfate | 0.40 | 0.10 |
| Mannitol | 207.60 | 51.90 |
| Cellulose, microcrystalline | 66.00 | 16.50 |
| Croscarmellose sodium | 10.00 | 2.50 |
| Magnesium stearate | 4.00 | 1.00 |
| Sub-total Core | 400.00 | 100.00 |
| Film-coat Opadry ® red* | 9.00 | — |
| Total Film coated tablet | 409.00 | — |

*Composition film coat: Hypromellose 2910, Macrogol 6000, titanium dioxide, talc, iron oxide red

TABLE 4
Composition formulation D

| | Dose strength | | |
|---|---|---|---|
| | 25 mg | 50 mg | 75 mg |
| | Drug load (%, wt/wt) | | |
| | 25.00 | 25.00 | 25.00 |
| Ingredient | [mg/tab] | [mg/tab] | [mg/tab] |
| Compound 1 (D90 ≤ 15 μm) | 25.00 | 50.00 | 75.00 |
| Hydroxypropyl cellulose | 3.00 | 6.00 | 9.00 |
| Sodium laurilsulfate | 0.10 | 0.20 | 0.30 |
| Mannitol | 51.90 | 103.80 | 155.70 |
| Cellulose, microcrystalline | 16.50 | 33.00 | 49.50 |
| Croscarmellose sodium | 2.50 | 5.00 | 7.50 |
| Magnesium stearate | 1.00 | 2.00 | 3.00 |
| Sub-total Core | 100.000 | 200.000 | 300.000 |
| Film-coat Aquapolish ® P^{#} | 5.000 | 8.000 | 10.000 |
| Total Film coated tablet | 105.00 | 208.00 | 310.00 |
| Dose strength | 100 mg | 125 mg | all |
| Drug load (%, wt/wt) | 25.00 | 25.00 | 25.00 |
| Ingredient | [mg/tab] | [mg/tab] | [%, wt/wt] |
| Compound 1 (D90 ≤ 15 μm) | 100.00 | 125.00 | 25.00 |
| Hydroxypropyl cellulose | 12.00 | 15.00 | 3.00 |
| Sodium laurilsulfate | 0.400 | 0.50 | 0.10 |
| Mannitol | 207.60 | 259.50 | 51.90 |
| Cellulose, microcrystalline | 66.00 | 82.50 | 16.50 |
| Croscarmellose sodium | 10.00 | 12.50 | 2.50 |
| Magnesium stearate | 4.00 | 5.00 | 1.00 |
| Sub-total Core | 400.00 | 500.00 | 100.00 |
| Film-coat Aquapolish ® P^{#} | 12.000 | 14.000 | — |
| Total Film coated tablet | 412.00 | 514.00 | — |

^{#}Composition film coat: Hypromellose 2910, hydroxypropylcellulose, calcium carbonate, isomalt, medium chain triglycerides, iron oxide yellow

The 5 mg tablets of formulations A and B were dull red, round, biconvex, bevel-edged film-coated tablets about 6 mm in diameter.

The 25 mg tablets of formulations A and B were dull red, oval, biconvex, film-coated tablets about 14×6.8 mm in length and width.

The 50 mg tablets of formulations A and B were dull red, oval, biconvex, film-coated tablets about 17.8×8.6 mm in length and width.

The 100 mg tablets of formulation C were dull red, oval, biconvex, film-coated tablets about 15×7 mm in length and width.

The tablets of formulation D had the following size, shape, and color:

| 25 mg | 50 mg | 75 mg | 100 mg | 125 mg |
|---|---|---|---|---|
| 6.5 mm | 11 × 5.5 mm | 13 × 6.3 mm | 15 × 7 mm | 16.2 × 7.9 mm |
| round | oval | oval | oval | oval |
| light yellow | light yellow | light yellow | light yellow | light yellow |
| or yellow | or yellow | or yellow | or yellow | or yellow |

The wet-granulation process used for preparing the tablets of formulations A, B, C and D was a fluid bed granulation process using the following granulation conditions:

Batch size 45 kg final blend:

| | | |
|---|---|---|
| Inlet air temperature | 80-100° | C. |
| Inlet air volume | 800-1400 | m³/h |
| Spray rate | 300-400 | g/min |

Batch size 100 kg final blend:

| | | |
|---|---|---|
| Inlet air temperature | 90° | C. |
| Inlet air volume | 1400-2300 | m³/h |
| Spray rate | 650-680 | g/min |

The respective wet-granulation processes can be summarized as follows.

### Formulation A

### Manufacture of Compound 1 Suspension (Step 1)

### Step 1.1 Preparation of the Vehicle

Hydroxypropylcellulose, sodium laurilsulfate, polysorbate 80 and methyl parahydroxybenzoate are added to purified water and mixed to produce a vehicle.

### Step 1.2 Preparation of the Suspension

Jet-milled Compound 1, D90≤15 μm, is added to the vehicle and stirred to obtain a suspension.

### Step 1.3 Milling

The suspension is milled in a wet milling process to obtain a nanosized Compound 1, target size D50<200 nm, suspension.

### Manufacture of Compound 1 Granulation Liquid (Step 2)

### Step 2.1 Preparation of the Binder Liquid

Hydroxypropylcellulose and purified water are mixed in a suitable vessel to obtain a binder liquid.

### Step 2.2 Preparation of the Granulation Liquid

While stirring the nanosized Compound 1 suspension obtained in Step 1, the binder liquid is added. Afterwards mannitol is added to obtain a granulation liquid.

### Manufacture of Compound 1 Granules (Step 3)

### Step 3.1 and Step 3.2 Pre-Heating and Granulating

Mannitol is pre-screened and pre-heated and then granulated with the granulation liquid obtained in Step 2 in a suitable fluid-bed granulator.

### Step 3.3 Drying

The obtained wet granules are dried in the fluid-bed granulator to obtain granules, dried.

### Step 3.4 Dry Screening

The dried granules are screened with a suitable screen.

### Manufacture of Compound 1 Final Blend (Step 4)

### Step 4.1 Main Blending

Microcrystalline cellulose, croscarmellose sodium and the pre-screened granules obtained in Step 3 are blended to obtain a main blend.

### Step 4.2 Pre-Screening

A part of the main blend is pre-screened with magnesium stearate.

### Step 4.3 Final Blending

The remainder of the main blend and the pre-screened material are blended to produce a final blend.

### Manufacture of Compound 1 Tablet Cores (Step 5)

### Step 5.1 Tablet Compression

The final blend obtained in Step 4 is compressed into tablet cores using a suitable tablet press.

### Manufacture of Compound 1 Film-Coated Tablets (Step 6)

### Step 6.1 Preparation of the Film-Coating Suspension

The film-coating mixture (Opadry® red) is dispersed in purified water by stirring in a suitable mixing vessel to prepare a film-coating suspension.

### Step 6.2 Film Coating

The tablet cores obtained in Step 5 are coated with the film-coating suspension by spraying in a drum coater to produce Compound 1 film-coated tablets.

### Formulations B and C

### Manufacture of Compound 1 Granulation Liquid (Step 1)

### Step 1.1 Preparation of the Granulation Suspension

Hydroxypropylcellulose, sodium laurilsulfate and mannitol (optionally pre-screened) are dissolved in purified water. Afterwards jet-milled Compound 1, D90≤15 μm, is suspended therein to obtain a granulation liquid.

### Manufacture of Compound 1 Granules (Step 2)

### Step 2.1 and Step 2.2 Pre-Heating and Granulating

Mannitol is pre-screened and pre-heated and then granulated with the granulation liquid obtained in Step 1 in a suitable fluid-bed granulator.

### Step 2.3 Drying

The obtained wet granules are dried in the fluid-bed granulator to obtain granules, dried.

### Step 2.4 Dry Screening

The dried granules are screened with a suitable screen.

### Manufacture of Compound 1 Final Blend (Step 3)

### Step 3.1 Main Blending

Microcrystalline cellulose, croscarmellose sodium and the pre-screened granules obtained in Step 2 are blended to obtain a main blend.

### Step 3.2 Pre-Screening

A part of the main blend is pre-screened with magnesium stearate,

### Step 3.3 Final Blending

The remainder of main blend and the pre-screened material are blended to produce a final blend.

### Manufacture of Compound 1 Tablet Cores (Step 4)

### Step 4.1 Tablet Compression

The final blend obtained in Step 3 is compressed into tablet cores using a suitable tablet press.

### Manufacture of Compound 1 Film-Coated Tablets (Step 5)

### Step 5.1 Preparation of the Film-Coating Suspension

The film-coating mixture (Opadry® red) is dispersed in purified water by stirring in a suitable mixing vessel to prepare a film-coating suspension.

### Step 5.2 Film Coating

The tablet cores obtained in Step 4 are coated with the film-coating suspension by spraying in a drum coater to produce Compound 1 film-coated tablets.

### Formulation D

### Manufacture of Compound 1 Granulation Liquid (Step 1)

### Step 1.0 Preparation of the Granulation Suspension

Hydroxypropylcellulose, sodium laurilsulfate and mannitol are dissolved in purified water. Afterwards jet-milled Compound 1, D90≤15 μm, is suspended therein to obtain a granulation liquid.

### Manufacture of Compound 1 Granules (Step 2)

Step 2.1 and Step 2.2 Pre-Heating and Granulating

Mannitol is pre-screened and pre-heated and then granulated with the granulation liquid obtained in Step 1 in a suitable fluid-bed granulator.

### Step 2.3 Drying

The obtained wet granules are dried in the fluid-bed granulator to obtain granules, dried.

### Manufacture of Compound 1 Final Blend (Step 3)

### Step 3.1 and Step 3.2 Main Blending

Microcrystalline cellulose, croscarmellose sodium and the dried granules obtained in Step 2 are blended, and then magnesium stearate is added to obtain a main blend.

### Step 3.3 and Step 3.4 Screening and Final Blending

The main blend is screened and finally blended to produce a final blend.

### Manufacture of Compound 1 Tablet Cores (Step 4)

### Step 4.1 Tablet Compression

The final blend obtained in Step 3 is compressed into tablet cores using a suitable tablet press.

### Manufacture of Compound 1 Film-Coated Tablets (Step 5)

### Step 5.1 Preparation of the Film-Coating Suspension

The film-coating mixture (Aquapolish® P) is dispersed in purified water by stirring in a suitable mixing vessel to prepare a film-coating suspension.

### Step 5.2 Film Coating

The tablet cores obtained in Step 4 are coated with the film-coating suspension by spraying in a drum coater to produce Compound 1 film-coated tablets.

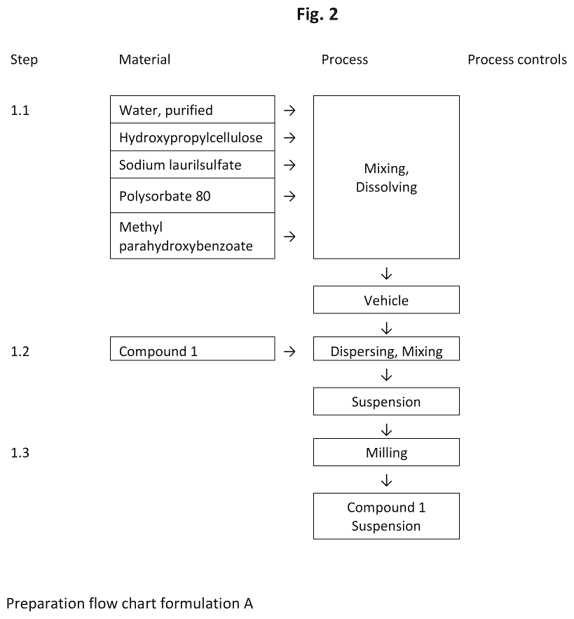

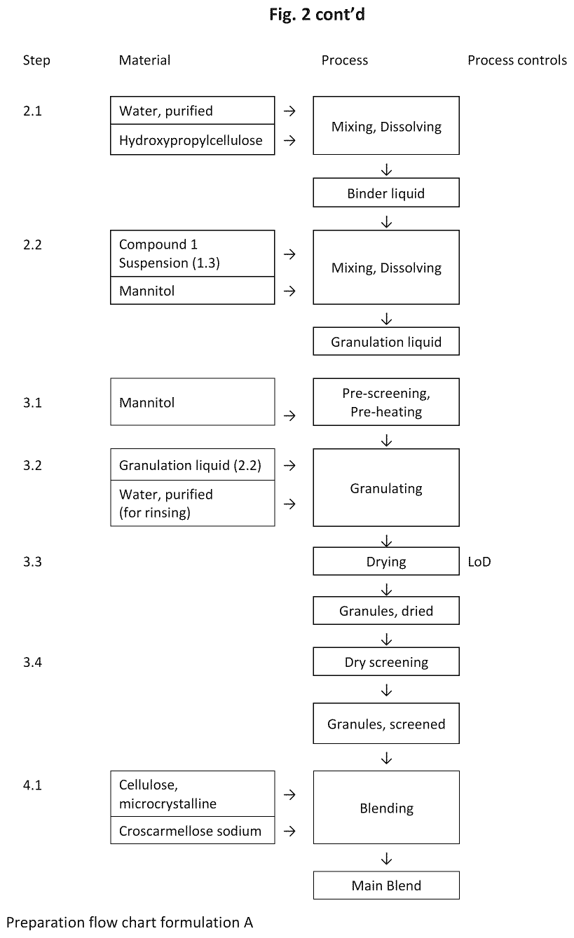

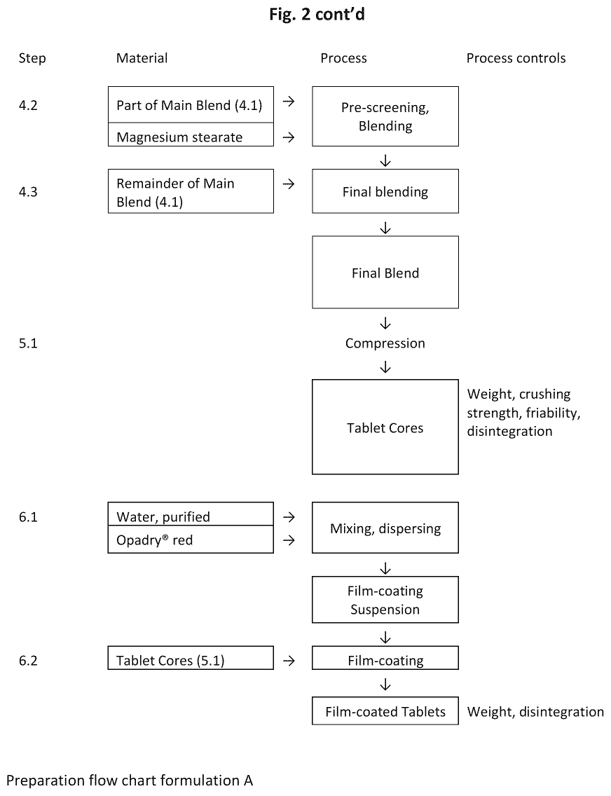

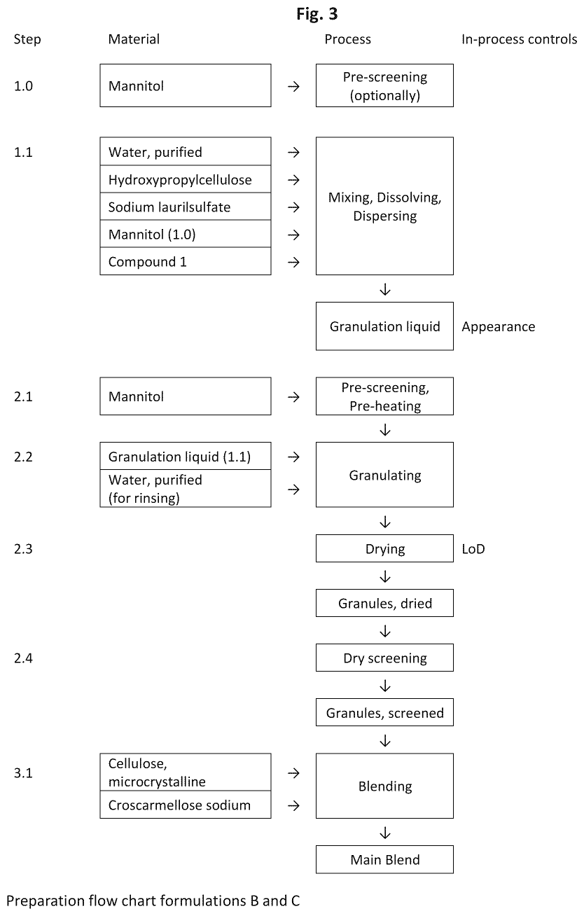

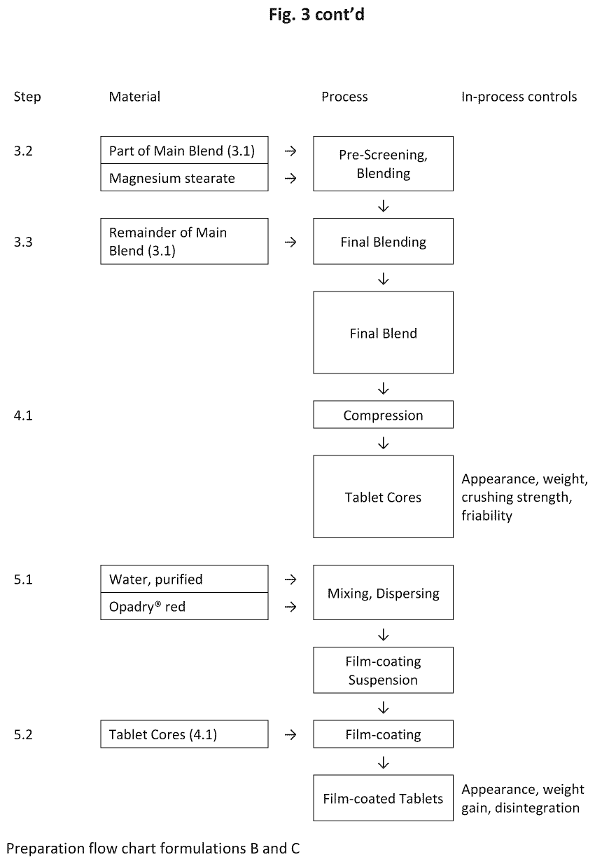

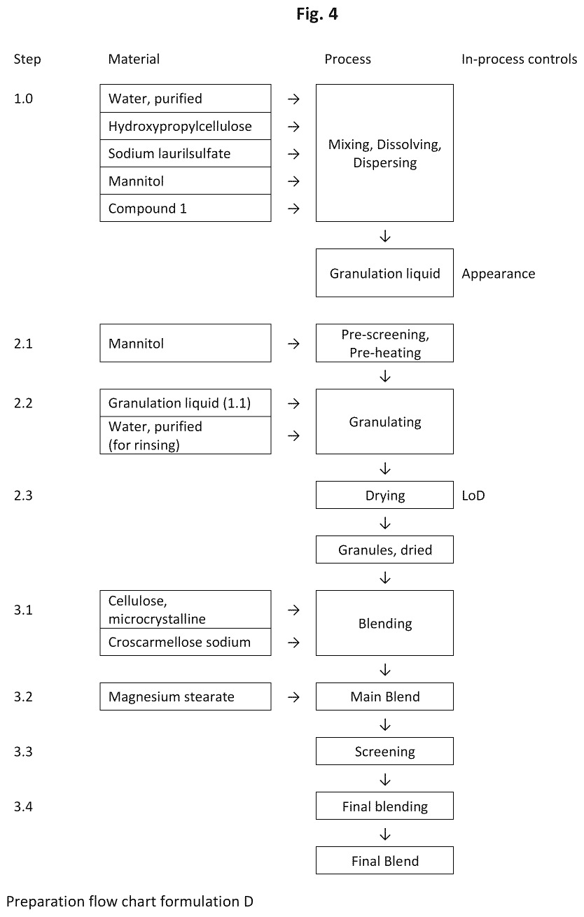

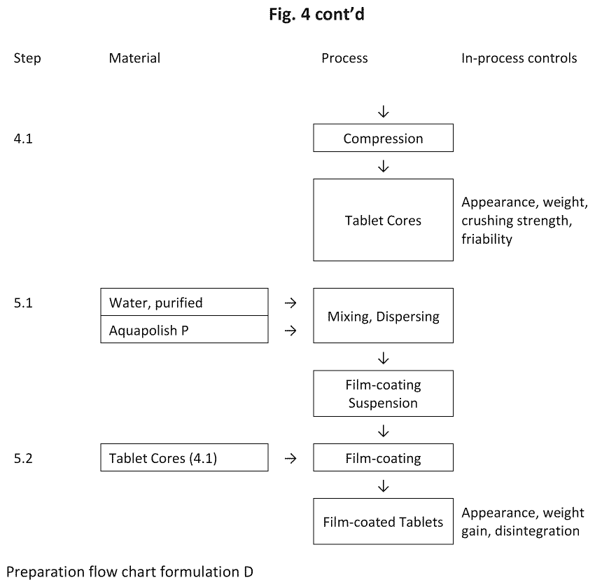

Flow charts of the corresponding preparation methods are shown in FIGS. 2, 3 and 4.

### Example 2 Bioavailability Testing

Two bioavailability studies (Study 1 and Study 2) were performed using the tablets of formulations A, B, and C. Differences in exposure (AUC) as well as food effect differences were investigated following oral administration under fed and fasted conditions in healthy male subjects.

Study details were as follows:

| Study design: | Open-label, single-dose, randomised, crossover | |
|---|---|---|
| | study | |
| Number of patients: | 24 | |
| Inclusion criteria: | Healthy male subjects, | |
| | Study 1: | Age 18 to 50 years (inclusive), |
| | Study 2: | Age 18 to 55 years (inclusive), |
| | Body mass index (BMI) 18.5 to 29.9 kg/m² | |
| | (inclusive) | |
| Investigational | Study 1: | Tablets of formulation A and |
| products: | | formulation B each containing |
| | | 50 mg Compound 1 per tablet |
| | Study 2: | Tablets of formulation B containing |
| | | 50 mg Compound 1 per tablet and |
| | | tablets of formulation C containing |
| | | 100 mg Compound 1 per tablet |

| | |
|---|---|
| Dose: | 100 mg (2 × 50 mg or 1 × 100 mg) |
| Mode of | Two 50 mg tablets oral at once or one 100 mg tablet |
| administration: | oral with 240 mL of water following a high-fat, |
| | high-calorie breakfast (fed), or |
| | Two 50 mg tablets oral at once or one 100 mg tablet |
| | oral after an overnight fast of at least 10 h (fasted) |
| Duration of | One day (single dose) for each treatment with a |
| treatment: | washout period of at least 17 days between the |
| | treatments |

Bioavailability (AUC_{0-∞}) was determined based on the geometric means (gMean) of the obtained plasma concentration data.

Tables 4a and 4b below show the results of the bioavailability studies. A sufficiently high AUC was obtained for all formulations both in the fasted and in the fed state.

TABLE 4a
Results of Study 1 on oral bioavailability and food effects

AUC_{0-∞} (nmol · h/L)
at doses of 100 mg

| | Formulation A | Formulation B |
|---|---|---|
| fasted | 13818 | 11715 |
| fed | 20752 | 20596 |
| ratio fed/fasted | 1.5 | 1.76 |

| | |
|---|---|
| ratio A/B fasted | 1.180 |
| ratio A/B fed | 1.008 |

TABLE 4b
Results of Study 2 on oral bioavailability and food effects

AUC_{0-∞} (nmol · h/L)
at doses of 100 mg

| | Formulation B | Formulation C |
|---|---|---|
| fasted | 9322 | 8159 |
| fed | 16066 | 16526 |
| ratio fed/fasted | 1.72 | 2.03 |

| | |
|---|---|
| ratio B/C fasted | 1.143 |
| ratio B/C fed | 0.972 |

The results of Study 1 and Study 2 confirm the favorable pharmacokinetic properties of the solid oral pharmaceutical compositions of the present invention with a high oral bioavailability and a fed/fasted ratio of 2.03 or below.

### Example 3 Dissolution Testing

Dissolution testing was performed with the 50 mg tablet cores and the final 50 mg film-coated tablets of formulation B under the following conditions:
0.1 M Hydrochloric acid pH 1+0.2% SL5, 900 mL, paddle, 190 rpm.

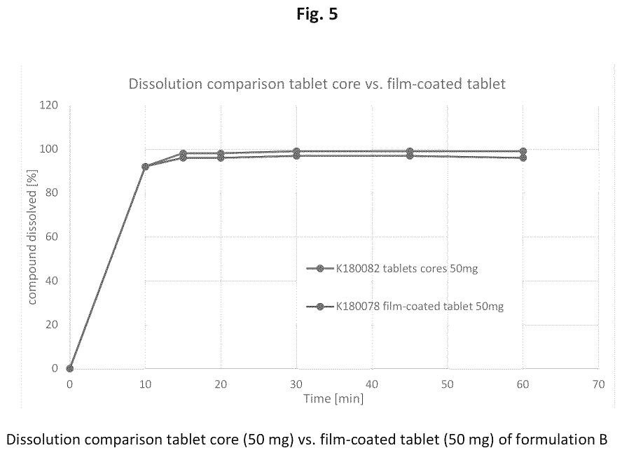

Results are shown in FIG. 5. Both the tablet cores and the final film-coated tablets showed an immediate release profile with a complete release of Compound 1 within less than 20 min.

### Example 4 Preparation of Compound 1

### List of Abbreviations:

APCI Atmospheric Pressure Chemical Ionization
abs. absolute
aq. aqueous
BHT 3,5-di-tert-butyl-4-hydroxytoluol
conc concentrated
DCM dichloromethane
DIPEA N-ethyl-diisopropylamine
DMAc dimethylacetamide
DMF dimethylformamide
DMSO dimethylsulfoxide
equiv. equivalents
ESI Electrospray Ionization
EtOAc ethyl acetate
g gram
h hour(s)
HOAc acetic acid
HPLC high performance liquid chromatography
iPr iso-propyl
kg kilogram
NMP N-methyl-2-pyrrolidon
NMR Nuclear Magnetic Resonance
MeCN acetonitrile
MeOH methanol
min minute(s)
mg milligram
mL milliliter
M Molar (mol/L)
TBABr tetra-n-butylammonium bromide
THF tetrahydrofuran

### NMR Method

NMR spectra were recorded on a Bruker AVANCE III instrument with a frequency of 600 MHZ for ¹H-NMR experiments and 150 MHz for 13C-NMR experiments, respectively, and using TopSpin 3.2 pl6 software for analysis. Chemical shifts are given in parts per million (ppm) downfield from internal reference trimethylsilane in 8 units. Selected data are reported in the following manner: chemical shift (multiplicity, coupling constants (J), number of hydrogens). Abbreviations are as follows: s (singulet), d (doublet), t (triplet), q (quartet), spt (septet), m (multiplet), br (broad).

### X-Ray Powder Diffraction (XRPD) Diagram

X-ray powder diffraction measurements were performed using a Bruker D8 Advance-diffractometer in reflection mode fitted with a LynxEye Position Sensitive detector, and a Cu-anode as X-ray source with CuKα1 radiation (λ=1.54060 Å, 40 kV, 40 mA). The standard error range for the 2-theta values is ±0.2°.

### Step 1: Preparation of 8-bromo-7-(4-chlorobenzyl)-3-methyl-3,7-dihydro-1H-purine-2,6-dione (Compound 2)

8-Bromo-3-methylxanthine (20.0 g, 81.6 mmol, 1.0 equiv.) and BHT (0.8 g, 3.6 mmol, 0.04 equiv.) are dissolved in dimethylacetamide (210 mL). The mixture is heated up to 85° C. A solution of 4-chlorobenzyl chloride (15.8 g, 97.9 mmol, 1.2 equiv.) in dimethylacetamide (20 mL) is added and rinsed with dimethylacetamide (10 mL). Diisopropylethylamine (11.1 g, 85.7 mmol, 1.05 equiv.) is added and rinsed with dimethylacetamide (10 mL). The reaction is stirred at 85° C. until the starting material is consumed (8-bromo-3-methylxanthine <0.3%). Optionally, further dosage of diisopropylethylamine (0.5 g, 4.1 mmol, 0.05 equiv.) may be performed to complete the reaction. After complete conversion, hydrochloric acid (4M, 0.8 g, 8.2 mmol, 0.1 equiv.) is added. The reaction solvent is removed partially via vacuum distillation (until a remaining volume of approx. 150 ml of the reaction mixture). Acetonitrile (150 mL) is added, and the product suspension is slowly cooled to 20° C. The product is isolated via filtration and the filter cake is washed two times with acetonitrile (50 mL). The isolated material is dried under reduced pressure at 50° C. giving Compound 2 (28.7 g, 78.0 mmol, 95% yield, 99.9% purity) as a colorless solid. Melting point: 270-271° C.

¹H NMR (DMSO-d₆) δ: 11.37 (s, 1H), 7.44 (d, J=8.5 Hz, 2H), 7.29 (d, J=8.5 Hz, 2H), 5.48 (s, 2H), 3.34 (s, 3H); 13C NMR (DMSO-d₆) δ: 154.0, 150.5, 149.3, 134.5, 132.6, 129.0, 128.7, 127.9, 108.6, 48.6, 28.5; HRMS (ESI): m/z 369, ([M+H]⁺, exp. 368.9763, calc. 368.9748).

### Step 2: Preparation of 8-bromo-7-(4-chlorobenzyl)-1-(3-hydroxypropyl)-3-methyl-3,7-dihydro-1H-purine-2,6-dione (Compound 3)

Compound 2 (20.0 g, 54.1 mmol, 1.0 equiv.) and sodium bicarbonate (6.8 g, 81.2 mmol, 1.5 equiv.), tetrabutylammonium bromide (0.8 g, 2.7 mmol, 0.05 equiv.) are suspended in dimethylacetamide (170 mL). The mixture is heated to 110° C. 3-chloro-1-propanol (7.7 g, 81.5 mmol, 1.5 equiv.) is added, and rinsed with dimethylacetamide (10 mL). Vacuum (200-400 mbar) is then applied. The reaction is stirred at 110° C. until the starting material is consumed (Compound 2<0.5%). After complete conversion, the reaction mixture is cooled to 80° C., filtered and rinsed with dimethylacetamide (30 mL). Water (160 mL) is added to the filtrate at 90° C., then sodium bicarbonate (0.5 g, 5.4 mmol, 0.1 equiv.) is added. The mixture is cooled to 70° C. and seed crystals (47 mg) are added. The crystal suspension is cooled to 40° C. over 60 min, heated to 70° C., kept for at least 15 min at 70° C. and cooled to 20° C. over 150 min and stirred for 1 h. The product is isolated via filtration and the filter cake is washed with water (160 mL). The isolated material is dried under reduced pressure at 60° C. giving Compound 3 (16.1 g, 37.7 mmol, 91% yield, 98.7% purity) as a colorless solid. Melting point: 148-149° C.

¹H NMR (DMSO-d₆) δ: 7.43 (d, J=8.5 Hz, 2H), 7.30 (d, J=8.5 Hz, 2H), 5.52 (s, 2H), 4.48 (t, J=5.2 Hz, 1H), 3.89-3.96 (m, 2H), 3.43-3.48 (m, 2H), 3.38 (s, 2H), 1.65-1.74 (m, 2H); 13C NMR (DMSO-d₆) δ: 153.5, 150.3, 147.9, 134.5, 132.6, 129.0, 128.7, 128.2, 108.2, 58.7, 48.7, 38.5, 30.8, 29.5; HRMS (ESI): m/z 427, ([M+H]⁺, exp. 427.0188, calc. 427.0167).

### Step 3: Preparation of 7-(4-chlorobenzyl)-1-(3-hydroxypropyl)-3-methyl-8-(3-(trifluoromethoxy)phenoxy)-3,7-dihydro-1H-purine-2,6-dione (Compound 1) [crude]

Compound 3 (20.0 g, 46.8 mmol, 1.0 equiv.), tetrabutylammonium bromide (0.8 g, 2.4 mmol, 0.05 equiv.) and sodium carbonate (3.5 g, 32.7 mmol, 0.7 equiv.) are suspended in N-methyl-2-pyrrolidone (135 mL). The mixture is heated to 50° C. 3-(Trifluoromethoxy)phenol (9.2 g, 51.7 mmol, 1.1 equiv.) is added and rinsed with N-methyl-2-pyrrolidone (10 mL). The mixture is heated to 120° C. and stirred at 120° C. under reduced pressure (200-400 mbar) until the starting material is consumed (Compound 3<1.0%). After complete conversion, the reaction mixture is cooled to 80° C., filtered and rinsed with N-methyl-2-pyrrolidone (15 ml). Acetonitrile (40 mL) is added. Water (110 mL) is added in at least 30 min. The mixture is cooled to 58° C. and seed crystals (20 mg) are added. The crystal suspension is consecutively cooled to 40° C., heated to 60° C., kept for at least 15 min at same temperature and cooled to 20° C. The product is isolated via filtration and the filter cake is washed with water (160 mL) and n-heptane (40 mL). The isolated material is dried under reduced pressure at 60° C. giving Compound 1 [crude] (22.1 g, 42.1 mmol, 90% yield, 98.5% purity) as a colorless solid. Melting point: 124-125° C.

¹H NMR (DMSO-d₆) δ: 7.56-7.63 (m, 1H), 7.49 (s, 1H), 7.40-7.45 (m, 5H), 7.32 (br d, J=8.3 Hz, 1H), 5.44 (s, 2H), 4.47 (t, J=5.3 Hz, 1H), 3.87-3.96 (m, 2H), 3.39-3.48 (m, 2H), 3.29 (s, 3H), 1.63-1.75 (m, 2H); 13C NMR (DMSO-d₆) δ: 153.8, 153.7, 152.1, 150.5, 148.6, 145.4, 135.1, 132.5, 131.3, 129.5, 128.7, 118.8, 118.2, 119.9, 113.1, 102.5, 58.7, 45.8, 38.3, 30.9, 29.5; HRMS (ESI): m/z 525 ([M+H]⁺, exp. 525.1151, calc. 525.1147).

### Step 4: Recrystallization of 7-(4-chlorobenzyl)-1-(3-hydroxypropyl)-3-methyl-8-(3-(trifluoromethoxy)phenoxy)-3,7-dihydro-1H-purine-2,6-dione (Compound 1)

Compound 1 [crude] (20.0 g, 38.1 mmol, 1.0 equiv.) is suspended in ethyl acetate (90 mL). The mixture is heated to 65° C., filtered, and n-heptane (100 mL) is added to the solution. The mixture is cooled to 53° C. and seed crystals (40 mg) are introduced. The crystalline suspension is stirred at least 60 min before n-heptane (100 mL) is being added and stirred another 60 min at 53° C. The suspension is cooled to 5° C. over 90 min, and stirred 120 min at 5° C. The product is isolated via filtration and the filter cake is washed with n-heptane (100 ml). The isolated material is dried under reduced pressure at 50° C. giving Compound 1 (18.9 g, 36.2 mmol, 95% yield, 99.8% purity) as a colorless solid. Melting point: 124° C.

¹H NMR (DMSO-d₆) δ: 7.56-7.63 (m, 1H), 7.49 (s, 1H), 7.40-7.45 (m, 5H), 7.32 (br d, J=8.3 Hz, 1H), 5.44 (s, 2H), 4.47 (t, J=5.3 Hz, 1H), 3.87-3.96 (m, 2H), 3.39-3.48 (m, 2H), 3.29 (s, 3H), 1.63-1.75 (m, 2H); 13C NMR (DMSO-d₆) δ: 153.8, 153.7, 152.1, 150.5, 148.6, 145.4, 135.1, 132.5, 131.3, 129.5, 128.7, 118.8, 118.2, 119.9, 113.1, 102.5, 58.7, 45.8, 38.3, 30.9, 29.5; HRMS (ESI): m/z 525 ([M+H]⁺, exp. 525.1150, calc. 525.1147).

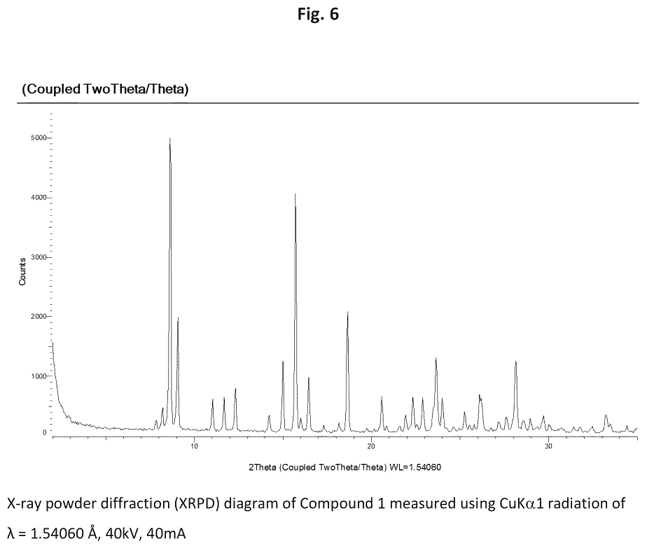

The recrystallized Compound 1 prepared according to Step 4 was analyzed by X-ray powder diffraction. Results as shown in FIG. 6 (XRPD-diagram) and Table 5.

TABLE 5
XRPD data for Compound 1

| No. | 2Theta, ° | Rel. Intensity |
|---|---|---|
| 1 | 8.7 ± 0.2 | 100 |
| 2 | 9.1 ± 0.2 | 40.8 |
| 3 | 11.7 ± 0.2 | 12.5 |
| 4 | 12.3 ± 0.2 | 13.5 |
| 5 | 15.0 ± 0.2 | 26.3 |
| 6 | 15.7 ± 0.2 | 84 |
| 7 | 16.5 ± 0.2 | 19 |
| 8 | 18.7 ± 0.2 | 43.7 |
| 9 | 23.7 ± 0.2 | 19 |
| 10 | 28.1 ± 0.2 | 25.4 |

1. A solid oral pharmaceutical composition comprising 7-(4-chlorobenzyl)-1-(3-hydroxypropyl)-3-methyl-8-(3-(trifluoromethoxy)-phenoxy)-3,7-dihydro-1H-purine-2,6-dione (Compound 1), a sugar alcohol and at least one further pharmaceutically acceptable excipient.

2. The solid oral pharmaceutical composition of claim 1, wherein the sugar alcohol is selected from mannitol, sorbitol, and xylitol.

3. The solid oral pharmaceutical composition of claim 1, wherein the sugar alcohol is mannitol.

4. The solid oral pharmaceutical composition of claim 1, wherein amount of the sugar alcohol is 40-80% [w/w].

5. The solid oral pharmaceutical composition of claim 1, wherein the at least one further pharmaceutically acceptable excipient comprises one or more selected from binders, fillers, disintegrants, glidants, lubricants, wetting agents, surfactants and preservatives.

6. The solid oral pharmaceutical composition of claim 1, wherein the at least one further pharmaceutically acceptable excipient comprises a binder.

7. The solid oral pharmaceutical composition of claim 6, wherein the binder is a cellulose derivative.

8. The solid oral pharmaceutical composition of claim 7, wherein the cellulose derivative is selected from methylcellulose, hydroxyethylcellulose and hydroxypropylcellulose.

9. The solid oral pharmaceutical composition of claim 8, wherein the cellulose derivative is hydroxypropylcellulose.

10. The solid oral pharmaceutical composition of claim 6, wherein amount of the binder is 1-5% [w/w].

11. The solid oral pharmaceutical composition of claim 6, wherein the sugar alcohol is mannitol and the binder is hydroxypropylcellulose.

12. The solid oral pharmaceutical composition of claim 1, wherein the composition is a granulate, a hard capsule or a tablet.

13. The solid oral pharmaceutical composition of claim 12, wherein the composition is a tablet, which is optionally coated.

14. The solid oral pharmaceutical composition of claim 1, wherein Compound 1 is present in an amount of 5-50% [w/w].

15. The solid oral pharmaceutical composition of claim 1, wherein Compound 1 is present as sole active ingredient.

16. The solid oral pharmaceutical composition of claim 1, wherein Compound 1 is incorporated in form of particles having a particle size of D90<100 μm.

17. The solid oral pharmaceutical composition of claim 1, wherein Compound 1 is incorporated in form of particles having a particle size of D50<500 nm.

18. The solid oral pharmaceutical composition of claim 1, wherein the composition comprises:

| | | |
|---|---|---|
| 5-50% | [w/w] | Compound 1, |
| 40-80% | [w/w] | sugar alcohol, and |

one or more further pharmaceutically acceptable excipient(s), wherein Compound 1, the sugar alcohol, and the one or more further pharmaceutically acceptable excipient(s) add up to a total of 100% [w/w].

19. The solid oral pharmaceutical composition of claim 18, wherein the sugar alcohol is mannitol.

20. The solid oral pharmaceutical composition of claim 1, wherein the composition is a tablet, and the tablet core comprises:

| | | |
|---|---|---|
| 5-50% | [w/w] | Compound 1 (incorporated in form of |
| | | micronized or nanosized particles), |
| 40-80% | [w/w] | sugar alcohol, |
| 0-10% | [w/w] | binder, |
| 0-30% | [w/w] | filler, |
| 0-10% | [w/w] | disintegrant, |
| 0-5% | [w/w] | glidant, |
| 0-5% | [w/w] | lubricant, |

and 0-1% [w/w] each of a wetting agent, a surfactant and a preservative, wherein the amount of Compound 1 and the respective excipients adds up to a total of 100% [w/w] and, wherein the tablet optionally is coated.

21. The solid oral pharmaceutical composition of claim 1, wherein the composition is a tablet, and the tablet core comprises:

| | | |
|---|---|---|
| 5-50% | [w/w] | Compound 1 (incorporated in form of |
| | | micronized or nanosized particles), |
| 40-80% | [w/w] | mannitol |
| 0-10% | [w/w] | hydroxpropylcellulose, |
| 0-30% | [w/w] | microcrystalline cellulose, |
| 0-10% | [w/w] | croscarmellose sodium, |
| 0-5% | [w/w] | colloidal silicon dioxide, |
| 0-5% | [w/w] | magnesium stearate, |
| 0-1% | [w/w] | sodium laurilsulfate, |
| 0-1% | [w/w] | Polysorbate 80, and |
| 0-1% | [w/w] | Parahydroxybenzoate, |

wherein the amount of Compound 1 and the respective excipients adds up to a total of 100% [w/w] and,
wherein the tablet optionally is coated.

22. The solid oral pharmaceutical composition of claim 13, wherein the tablet is coated, and the coating is free of titanium dioxide.

23. The solid oral pharmaceutical composition of claim 1 prepared by a wet granulation method, wherein Compound 1 preferably is suspended in the granulation liquid.

24. A method for preparing the solid oral pharmaceutical composition of claim 1, wherein the method is a wet granulation method.

25. The method of claim 24, wherein the method is a fluid bed granulation method and Compound 1 is suspended in the granulation liquid.

26. The method of claim 24, wherein the method comprises the following steps:
preparing a granulation liquid containing Compound 1 in micronized or nanosized form, a first portion of the sugar alcohol, and the at least one further pharmaceutically acceptable excipient, preferably a binder;
granulating the second portion of the sugar alcohol with the granulation liquid in a suitable fluid-bed granulator;
drying the wet granules in the fluid-bed granulator to obtain dry granules;
optionally, screening the dried granules with a suitable screen;
combining the granules with one or more selected from fillers, disintegrants, glidants, and lubricants to obtain a premix, optionally screening the premix, and blending the optionally screened premix to obtain a blend;
compressing the blend into tablet cores using a suitable tablet press; and
further optionally, coating the tablet cores with a previously prepared film-coating suspension by spraying to produce Compound 1-containing film-coated tablets.

27. The method of claim 26, wherein the at least one further pharmaceutically acceptable excipient contained in the granulation liquid includes a binder and one or more of a wetting agent, a surfactant and a preservative, preferably a wetting agent and/or a surfactant.

28. A granulation liquid containing Compound 1 in micronized or nanosized form, a sugar alcohol, at least one further pharmaceutically acceptable excipient, preferably a binder, and, optionally, one or more of a wetting agent, a surfactant and a preservative, and water.

29. A method for treatment of a condition in a subject, comprising administration to the subject of a solid oral pharmaceutical composition according to claim 1.

30. The method of claim 29, wherein the condition is selected from a neuropsychiatric disorder, e.g., major depressive disorder (MDD) and post-traumatic stress disorder (PTSD), a neurodegenerative disorder, nephropathy, and seizure disorder.
